SEQUENCE LISTING (2 sequences)

SEQ ID NO: 1 - DNA, 1368 bp, Artificial Sequence
  Description of Artificial Sequence Synthetic polynucleotide
atgcgtgacgatgcgccgaattcctgggagtcgcgggccgacgccttctccctctacggtttcactgacctgccctcggtgcaccggcgcggcgctgtggtgctgacgcatggcacggggccctacgttcacgacgttcacgggcgcgcctatctggatgccaattcggggctttggaacatggtggccgggttcgaccatccgggtctgatcgaggccgccaaggcgcagtatgaccgctttccgggctatcacgccttcttcgggcggatgtcggaccagacggtgatgctgtcggagaagctcgtggaggtctcgcccttcgaccgcggccgggtgttctacaccaactcgggctccgaggcgaacgacacgatggtcaagatcctgtggttcctcggggcggccgaagggcatcccgagcggcgcaagatcatcacccgcgtgaacagctatcacggcgtcaccgccgtctcggcctcgatgacgggcaagccctacaacagcctgttcggcctgccgctgccgggcttcatccatgtgggctgcccgcactactggcgcttcgggcaggagggggaaagcgaggccgccttcacccagcgcctcgcgcgcgaacttgaagccaccatcatcaaggaggggccggacacgatcgcgggcttcttcgccgagcccgtgatgggtgccggtggggtgatcccgccgtcggagggctatttccaggccatccagccggtcctgaaccgctacggcatccctctgatcgcggacgaggtgatctgcggcttcgggcggaccggcaacacctggggctgccagacctacgacttcgtgccggacgcgatcatcagctcgaagaacatcaccgcgggcttcttcccgatgggagcggtgatccttggggcggagctgtcggatcgggtgcaggcggccgccgaggcggtcgaggagttcccgcacggcttcaccgcctcgggccatccggtgggctgcgccatcgcgctaaaggccatcgacgtggtgatgaacgaggggctggccgagaatgtgaagcggctgacgccgaagttcgaggcggggctggcccatctggccgaacatccgaacatcggcgagtggcgcggcaagggtctgatgggggcgcttgaggcggtgaaggacaaggccaccaagacccccttccccggcgacctctcggtgtcggagcggatcgccaacacctgcaccgatcacgggctcatctgccggccgctgggccagtcgatcgtgctctgcccgcccttcatcatgacggaggcgcagatggacgagatgttcgacaagctgggcgccgcgctaaaaaaggtcttcgcagaggtcgcctga

SEQ ID NO: 2 - DNA, 1431 bp, Artificial Sequence
  Description of Artificial Sequence Synthetic polynucleotide
atgggcctgactgtgcagaaaatcaactgggaacaggtcaaagagtgggaccgtaaatatctgatgcgcaccttcagcactcagaacgaataccagccggtaccaatcgagagcaccgaaggcgattatctgatcatgccggacggtactcgtctgctcgatttcttcaaccagctgtactgcgtgaacctgggtcagaaaaaccagaaagtgaacgctgcgattaaagaagcgctggaccgttacggttttgtgtgggacacctacgcgactgactacaaagccaaagccgcaaaaattattatcgaagacatcctgggtgacgaagattggccgggtaaagtacgctttgtgagcaccggcagcgaagctgtagaaactgccctgaacatcgcacgtctgtataccaaccgtccgctggttgtaacccgcgaacacgattatcacggttggactggtggtgcagctacggttactcgtctgcgtagctatcgtagcggcctggttggcgaaaacagcgagagcttcagcgcgcagatcccgggtagcagctataacagcgctgttctgatggcgccgagcccaaacatgttccaggatagcaacggcaactgcctgaaagacgaaaacggtgaactgctgagcgtcaaatacacccgccgtatgattgagaactacggtccggaacaggttgcagcggtcattacggaagttagccagggtgcgggcagcgctatgccaccgtacgaatacattccgcagatccgcaaaatgaccaaagaactgggtgtgctgtggatcaacgacgaggttctgaccggtttcggtcgcacgggtaaatggttcggctaccagcactatggtgtgcagccggacattatcactatgggtaaaggcctgagcagcagcagcctgccggcaggtgcggttgttgtaagcaaagagatcgctgcctttatggacaaacaccgttgggaaagcgtgagcacctatgcaggccatccagttgctatggctgcagtatgtgccaacctcgaagtcatgatggaagagaacctggttgagcaggccaaaaacagcggcgaatatatccgcagcaaactggagctgctccaggagaaacacaaaagcatcggcaacttcgatggctacggtctgctgtggatcgtggatatcgtgaacgcgaaaaccaaaaccccgtacgtcaaactggaccgtaacttcacgcatggcatgaacccgaaccagattccgacccagatcatcatgaaaaaagcgctggaaaaaggcgttctgattggcggcgttatgccgaacactatgcgtatcggtgctagcctgaacgtaagccgcggtgatatcgataaagcgatggacgcactggattacgctctggattacctggaaagcggtgaatggcagcagagctaa

(19) United States
(12) Patent Application Publication
(10) Pub. No.: US 2017/0101654 A1
(43) Pub. Date: Apr. 13, 2017

(54) ENZYMATIC SYNTHESIS OF OPTICALLY ACTIVE CHIRAL AMINES

(71) Applicant: Embio Limited, Powai (IN)

(72) Inventors: Arumugam Govind SWAMINATHAN, Mumbai (IN); Sunil Vaman JOSHI, Maharashtra (IN)

(73) Assignee: Embio Limited, Powai (IN)

(21) Appl. No.: 15/218,270

(22) Filed: Jul. 25, 2016

Related U.S. Application Data
(63) Continuation-in-part of application No. 14/008,725, filed on Dec. 18, 2013, now abandoned.

(30) Foreign Application Priority Data
Aug. 16, 2011 (IN) 2312/MUM/2011

(51) Int. Cl. C12P 13/00

(52) U.S. Cl. CPC C12P 13/001; C12Y 206/00

(57) ABSTRACT

The present invention relates to a method of enantioselective enzymatic transamination of (1R)-1-Hydroxy-1-Phenylacetone (R-PAC) to a chiral amine 1R, 2S-Norephedrine in the presence of an isopropylamine catalyzed by enantioselective transaminase. Isopropylamine is converted to acetone in the process. The transaminase used is in completely purified form, partially purified form or as whole cells. The source is microbial cells, which are genetically engineered. In the present invention, the enzyme is expressed in E. coli and used preferentially as a suspension of native cells. Transaminase comprising polypeptide sequence is obtained from Rhodobacter sphaeroides and expressed in E. coli. The nucleotide sequence of transaminase is expressed in an expression vector system pIEP/Kan/IEP AT12, which is incorporated in E. coli. The yield of 1R, 2S-1-norephedrine is greater than 87% and de % is greater than 99%. The enantioselective transamination process is cost-effective and environment-friendly in addition to providing the amine in high yield and enantioselectivity.

### SEQUENCE LISTING

The attached Sequence Listing is hereby incorporated by reference herein.

### TECHNICAL FIELD OF THE INVENTION

The present invention discloses a method for production of optically active chiral amine from alpha hydroxy ketone using an enzyme transaminase as a biocatalyst. More particularly, the present invention relates to the method of enantioselective enzymatic amination of (1R)-1-Hydroxy-1-Phenylacetone (R-PAC) to a chiral amine 1R, 2S-1-Norephedrine in the presence of an amino donor isopropylamine catalyzed by an enantioselective transaminase and resulting in formation of a ketone as a byproduct of the reaction.

### BACKGROUND OF THE INVENTION

Chiral amine plays an important role in the stereoselective organic synthesis, which is applicable in pharmaceutical and chemical industry. Chiral amines are generally used as resolving agents, building blocks, intermediates or chiral auxiliaries for the preparation of various physiologically and pharmaceutically active substances. In a great number of the various applications of chiral amines, only one particular optically active form either the (R) or the (S) enantiomer exhibits the desired physiological activity. Thus, it is crucial to provide a method for preparation of chiral amines in an optically active form.

Majority of drugs are amines or at least comprise functional groups derived from amines and these molecules are chiral and non-racemic in nature. Hence, production of different biological molecules derived from natural or synthetic sources relies on the development of efficient methods for production of chiral amines. Further, enantioselective synthesis of chiral amines plays an important role.

Norephedrine or 2-amino-1-phenyl-1-propanol is a naturally occurring alkaloid found in Chinese herb ‘Ma Huang’ or Ephedra, also an optically active amine. It is isolated from the herb along with l-ephedrine and other alkaloids. Apart from the natural source, norephedrine is also synthesized by various chemical methods such as catalytic reductive amination, catalytic hydrogenation etc. One of the serious drawbacks associated with the chemical synthesis is that it does not provide diastereoselectivity and hence a significant quantity of diastereomer is also obtained by such methods.

There are different methods available for preparation of norepinephrine. One of the methods is by resolution of dl-phenylpropanolamine.

The PCT Application No. PCT/EP2007/001222 titled “Process for the preparation of optically active chiral amines” discloses a process for preparation of optically active chiral amines comprising the steps of providing an amino acceptor and an amino donor, reacting the amino acceptor and the amino donor with a transaminase, in particular (R)- or (S)-selective transaminase and finally obtaining the desired optically active chiral amine and an α-ketone by-product. The optically active chiral amine obtained finally is isolated and purified from the reaction mixture. The invention discloses a process for the asymmetric synthesis of chiral amines by using transaminase to facilitate transamination of an amino group from an amino donor to an amino acceptor, thereby forming the desired product. Depending on the enantiopreference of the specific transaminase used, an optically active chiral amine of the desired optical configuration, i.e. either the (R) or (S) enantiomer, is obtained. Thus, using a (S)-selective-transaminase for the asymmetric synthesis generates the desired (S) enantiomer of the chiral amine while using in another embodiment of the present invention an (R)-selective-transaminase generates the desired (R)-enantiomer. However, the drawback associated with the prior art is the yield of the final product and the method is generic, which lacks the synthesis of specific chiral amine.

The U.S. Pat. No. 6,133,018 A titled “Enzymatic synthesis of chiral amines using 2-amino propane as amine donor” discloses an enzymatic synthesis of chiral compounds containing an amino group e.g., chiral amines. The invention constitutes the improvement in-the known stereoselective synthesis of a chiral amine in which a ketone is brought into contact with a transaminase in the presence of an amino donor and utilizing 2-aminopropane as the amine donor. The invention discloses the preparation of (S)-1-methoxy-2-aminopropane in which methoxyacetone is reacted with a transaminase in the presence of 2-aminopropane as an amine donor, permitting the reaction to continue until a substantial amount of methoxyacetone is converted to (S)-1-methoxy-2-aminopropane (and 2-aminopropane is simultaneously converted to acetone) and isolating the (S)1-methoxy-2-aminopropane thus formed. However, the invention is associated with less yield of the final product and is not cost effective.

The publication titled “Alpha-Amino acids as chiral educts for asymmetric products. Amino acylation with N-acylamino acids” by Thomas F. Buckley III, Henry Rapoport discloses an approach for a stereospecific synthesis of 1-erythro-2-amino-1-phenyl-1-propanol from chiral precursors. Alpha-N-Acylamino acids are developed as useful reagents for the preparation of optically pure-alpha-aminoalkyl aryl ketones. The protection of the amino group as either the ethoxycarbonyl or benzenesulfonyl derivative allows alanine to serve as an effective educt for the chirally specific synthesis of a variety of structures containing the phenylethylamine backbone. Benzene undergoes Friedel-Crafts acylation with the N-acylamine acid chloride. The catalyst complexation with oxygenated aromatics, however, prohibits acylation of aryl ethers. As an example, optically pure ephedrines and amphetamines are directly synthesized without recourse to resolution since the chirality of the amino acid educt is entirely conserved throughout the process. However, the invention does not disclose the use of stereospecific transaminase.

Consequently, review of methods in the prior arts shows that all the above stated methods suffer from at least one of the following drawbacks such as cost and recyclability of hydrogenation catalyst, cost and recyclability of resolving agents, poor diastereo- and enantioselectivity in reduction reactions, cost and availability of chiral precursors, chiral auxiliaries or chiral catalysts, generation of gaseous, liquid and solid effluents, which may be hazardous.

In view of the mentioned drawbacks in the prior arts, there is a need to develop a method for the preparation of 1-erythro-2-amino-1-phenyl-1-propanol (1-Norephedrine) that bypasses the above limitations and is more efficient in terms of yield and resolution and at the same time is cost-effective for which an enzymatic approach is suitable to the above mentioned problems.

### SUMMARY OF THE INVENTION

The present invention relates to a method of enantioselective enzymatic amination of (1R)-1-hydroxy-1-phenylacetone to a chiral amine 1R, 2S-1-Norephedrine in the presence of an amino donor isopropylamine catalyzed by enantioselective transaminase and in the process, reacted isopropylamine is converted to acetone.

The present invention provides a process for the synthesis of optically active chiral amines by using a transaminase for the transamination of an amino group from an amino donor to a keto substrate acting as amino acceptor, thereby forming the desired product. (1R)-1-hydroxy-1-phenylacetone is converted to 1R, 2S-Norephedrine through enantioselective enzymatic amination in the presence of transaminase as biocatalyst. For instance, the S-specific transaminase enzyme is capable of catalyzing the transfer of an amino group from an amino donor to a keto substrate, thereby forming S-specific chiral amine. Similarly, a R-specific transaminase enzyme catalyses the transfer of an amino group from an amino donor to a keto substrate, thereby forming R-specific chiral amine. The transaminase enzyme catalyses the transamination reaction by transferring an amino group from the amino donor to an amino acceptor. The products of this reaction are an amine product and an amino acceptor (ketone) byproduct.

The transaminase used is the present invention is in completely purified form, partially purified form or in microbial cells, in which the enzyme is expressed and is isolated from a genetically engineered organism. In the present invention, the enzyme is expressed in Escherichia coli and used as a suspension of native cells.

Transaminase comprising polypeptide sequence is obtained from Rhodobacter sphaeroides and expressed in E. coli. The nucleotide sequence of the transaminase is expressed in an expression vector system pIEP/Kan/IEP AT12, which is incorporated in E. coli. The E coli colonies transformed with the expression constructs are cultivated in 200 ml of Luria-Bertani (LB) medium. The expression of recombinant protein is induced by adding isopropylthiogalactoside (IPTG) at a concentration of 0.1 mM. After 16 hours of induction at 25° C. and 220 rpm, the cells are harvested and frozen at −20° C.

The transaminase reaction is carried out in a single phase system having the cells containing the enzyme suspended therein. The reaction is carried out as a conventional batch reaction under stirring. 0.5% by weight to 1.5% w/v of (1R)-1-hydroxy-1-phenylacetone is reacted with amino donor in the presence of transaminase. The aqueous portion of the reaction mixture also contains a buffer such as potassium phosphate, sodium phosphate or Tris-Hydrochloride (HCl) buffer. Co-substrates are added to the reaction mixture for the supply of the amino group. Further, a cofactor pyridoxal-5-phosphate is also added to the reaction. The pH and the temperature of the reaction mixture are maintained in the range of 6 to 10 and in the range of 25° C. to 35° C. respectively. Finally, the reaction is allowed to carried out for a period of 20 to 28 hours to result in the formation of 1R, 2S-1-Norephedrine.

After completion of the reaction, the percentage of 1R, 2S-1-Norephedrine is greater than 99.9%. The enantioselective transamination process is cost-effective and environment-friendly in addition to providing the amine in high yield and very high enantioselectivity.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 illustrates the chemical formula of 1R, 2S-1-Norephedrine.

FIG. 2 illustrates the chemical formula of (1R)-1-hydroxy-1-phenylacetone.

FIG. 3 illustrates a schematic representation of enantioselective enzymatic amination of (1R)-1-hydroxy-1-phenylacetone to a chiral amine 1R, 2S-1-norephedrine.

FIG. 4 illustrates the construction vector expressed in E. coli for the production of transaminase.

FIG. 5 illustrates a method of enzymatic synthesis of a chiral amine 1R, 2S-Norephedrine from (1R)-1-hydroxy-1-phenylacetone.

FIG. 6 illustrates the yield of the 1R, 2S-Norephedrine.

### DETAILED DESCRIPTION OF THE INVENTION

In order to more clearly and concisely describe and point out the subject matter of the claimed invention, the following definitions are provided for specific terms which are used in the following written description.

The term “Enantiomer” refers to one of two stereoisomers that are mirror images of each other and are non-superimposable on each other.

The term “Stereospecificity” refers to a property of a reaction mechanism that leads to different stereoisomeric reaction products from different stereoisomeric reactants or which operates on only one of the stereoisomers.

The term “Transaminase” refers to a polypeptide having an enzymatic capability of transferring an amino group (NH₂), a pair of electrons, and a proton from a primary amine to a carbonyl group (C═O) of an acceptor molecule. Transaminases as used herein include non-naturally occurring engineered polypeptide generated by human manipulation.

The term “Keto substrate”, “Keto” “Ketone” and “Amino acceptor” refers to a carbonyl (keto, or ketone) compound, which accepts an amino group from a donor amine.

The term “Amino donor”, “Amine donor” and “Donor amine” refers to any amino acid or amine that reacts with a transaminase and a ketone to produce desired amine product and a ketone by product.

The term “Recombinant” or “engineered” or “non-naturally occurring” refers to a cell, nucleic acid, or polypeptide, refers to a material, or a material corresponding to the natural or native form of the material, that has been modified in a manner that would not otherwise exist in nature, or is identical thereto but produced or derived from synthetic materials and by manipulation using recombinant techniques.

The present invention relates to a production of optically active chiral amine from alpha hydroxy ketone using enzyme transaminase as the biocatalyst. More particularly, the present invention relates to the method of enantioselective enzymatic amination of (1R)-1-hydroxy-1-phenylacetone to a chiral amine 1R, 2S-1-Norephedrine in the presence of the amino donor isopropylamine catalyzed by enantioselective transaminase and reacted isopropylamine is converted to acetone.

(1R)-1-hydroxy-1-phenylacetone is converted to 1R, 2S-1-Norephedrine through enantioselective enzymatic amination in the presence of transaminase as biocatalyst. Thus, the present invention provides a process for the synthesis of optically active chiral amines by using a transaminase for the transamination of an amino group from an amino donor to a keto substrate acting as amino acceptor, thereby forming the desired product. Depending on the enantiopreference of the specific transaminase used, an optically active chiral amine is obtained. For instance, the S-specific transaminase enzyme is capable of catalyzing the transfer of an amino group from an amino donor to a keto substrate, thereby forming S-specific chiral amine. Similarly, an R-specific transaminase enzyme catalyses the transfer of an amino group from an amino donor to a keto substrate, thereby forming R-specific chiral amine.

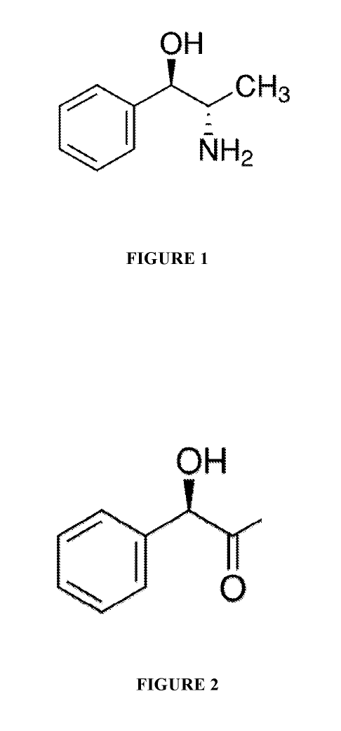

FIG. 1 illustrates the chemical formula of 1R, 2S-1-Norephedrine. 1R, 2S-1-Norephedrine is a chiral amino alcohol, which is produced by the present invention through enantioselective enzymatic transamination of alph hydroxy ketone, (1R)-1-hydroxy-1-phenylacetone.

FIG. 2 illustrates the chemical formula of (1R)-1-hydroxy-1-phenylacetone. (1R)-1-hydroxy-1-phenylacetone is subjected to enantioselective enzymatic transamination to produce chiral amine 1R, 2S-1-norephedrine.

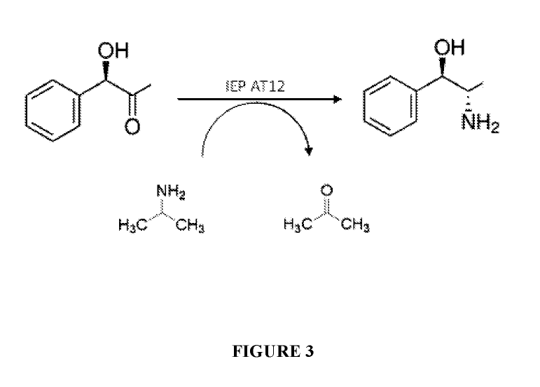

FIG. 3 illustrates a schematic representation of enantioselective enzymatic amination of (1R)-1-hydroxy-1-phenylacetone to a chiral amino alcohol 1R, 2S-1-Norephedrine. (1R)-1-hydroxy-1-phenylacetone in the presence of an amino donor is converted into 1R, 2S-Norephedrine in a reaction mixture comprising the enzyme transaminase as a biocatalyst, pyridoxal-5-phosphate as a cofactor along with a suitable buffer. The transaminase enzyme described in the present invention catalyses the transamination reaction by transferring an amino group from the amino donor to the amino acceptor also called as ketone substrate. The products of this reaction are an amine product and an amino acceptor (ketone) byproduct.

The amino donor is a molecule capable of providing an amino group to an amino acceptor or keto substrate using enzyme transaminase. In particular, the amino donor used in the present invention is an amine or amino acid. The amino acceptor is a molecule capable of accepting an amino group transferred from an amino donor by a transaminase. In particular, the amino acceptor exhibits ketone functionality.

The enzyme transaminase used in the present invention is isolated from expressed in a genetically engineered organism. The transaminase used is in completely purified form, partially purified form or in microbial cells, in which the enzyme is expressed. In the present invention, the enzyme is expressed in Escherichia coli and used as a suspension of native cells.

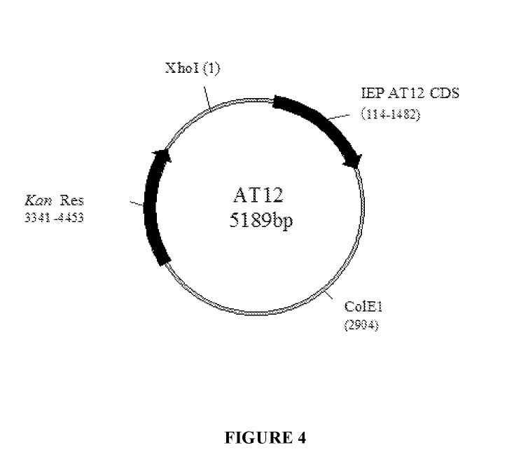

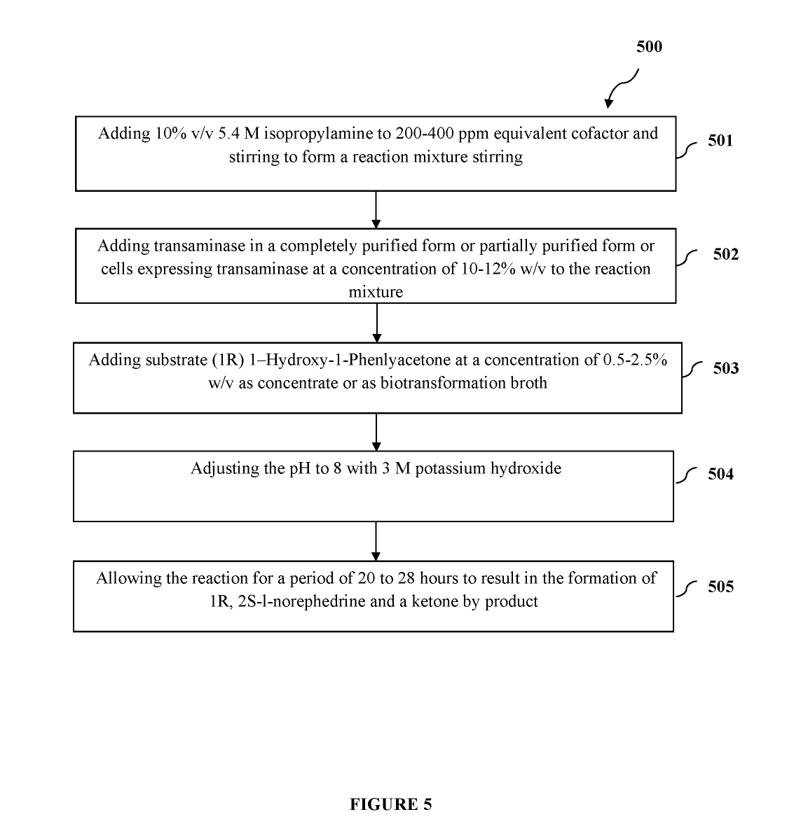

FIG. 4 illustrates the construction vector expressed in E. coli for the production of transaminase. The polypeptide sequence of transaminase is obtained from Rhodobacter sphaeroides in useable quantities by conventional procedures. Transaminase is expressed in a vector and further introduced in a suitable host organism, which is capable of expressing the desired gene. The preferred organism for the expression of transaminase is E. coli. E. coli is a rod shaped Gram-negative bacterium with peritrichous flagella and is a facultative anaerobe by nature. The nucleotide sequence of the transaminase is expressed in an expression vector system pIEP/Kan/IEP AT12, which is incorporated in E. coli. The E coli colonies transformed with the expression constructs are cultivated in 200 ml of LB medium. LB medium comprises 1% tryptone, 0.5% yeast extract and 1% sodium chloride with 50 μg/ml of kanamycin until an optical density of 0.5 measured at 550 nm is achieved. E. coli is allowed to grow at the temperature of 35° C. The expression of recombinant protein is induced by adding isopropylthiogalactoside (IPTG) at a concentration of 0.1 mM. After 16 hours of induction at 25° C. and 220 rpm, the cells are harvested and frozen at −20° C.

The transaminase expressed by transformed E. coli is extracted from E. coli cells in partially or completely purified form for use in the process or is also utilized in the cells themselves, which is in a native, permeabilized or lysed state.

The transaminase isolated from E. coli is SEQ ID NO: 1 or DSM 26761 comprising an amino acid sequence having at least 80% of the amino acids identical with the amino acid sequence SEQ ID NO: 2 i.e. isolated from Arthobacter and used as a reference. It is also found that the polypeptides comprising one of amino acid sequences SEQ ID NO: 1 or DSM 26761 a polypeptides comprising an amino sequence, which is identical by at least 80%, preferably at least 90% to the amino acid sequences SEQ ID NO: 2 and possessing transaminase activity is used for amination of (1R)-1-hydroxy-1-phenylacetone in the presence of an amino donor to 1R, 2S-1-norephedrine with high rate of conversion and with high enantiomeric selectivity.

In the present invention, in order to determine the percent identity of two polypeptides, the sequences are aligned for optimal comparison purposes. The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=# of identical positions/total # of positions (e.g., overlapping positions×100).

The “percent identity” of the two amino acid sequences is determined using the algorithm of Karlin and Altschul (Proc. Natl. Acad. Sci. USA 90:5873-5877, 1993). Such an algorithm is incorporated into the BLAST program of Altschul et al. (J. Mol. Biol. 215:403-410, 1990). While utilizing BLAST and Gapped BLAST programs, the default parameters of the respective programs are used.

The culture DSM 26761 has been deposited in IDA authority, DSMZ GmbH, Inhoffenstrasse 7B, 38124 Braunschweig Germany.

The transaminase reaction is carried out in a single phase system having the cells containing the enzyme suspended therein. The reaction is carried out as a conventional batch reaction. One of the significant advantages of the enantioselective enzymatic amination for commercial applications is that it is amenable to reaction without purification of substrate.

FIG. 5 illustrates a method of enzymatic synthesis of a chiral amine 1R, 2S-Norephedrine (1R)-1-hydroxy-1-phenylacetone. The method (500) starts at the step (501) of adding of an amine donor to a cofactor and stirring to form a reaction mixture. The present invention discloses the use of 5.4 M isopropylamine as an amino donor, at a concentration of 10% v/v in reaction mixture. Pyridoxal phosphate is added as a cofactor at a concentration of 200-400 ppm to the reaction mixture. At step (502), transaminase is added in the completely purified form or partially purified form or E. coli cells expressing transaminase is added at a concentration of 10-12% w/v to the reaction mixture. The suspension is the aqueous portion of the reaction mixture and also contains a buffer such as potassium phosphate, sodium phosphate or Tris-Hydrochloride (HCl) buffer or it also the biotransformation broth as such containing the substrate. At step (503), the substrate (1R) 1-hydroxy-1phenylacetone at a concentration of 0.5-2.5% w/v either as concentrate or as biotransformation broth is added. At step (504), pH is adjusted to 8 with 3 M potassium hydroxide. At step (505), the reaction is allowed to proceed for a period of 20 to 28 hours, by continuously stirring and the temperature is maintained between 25° C.-35° C., resulting in the formation of 1R,2S-Norephedrine and a ketone preferably acetone byproduct from used isopropylamine.

The buffer further comprises ions for the stabilization of the enzyme such as polyol selected from a group comprising glycerol, sorbitols, sulfur compounds such as 1,4-DL-dithiothreitol, glutathione, cysteine, peptides and detergents such as Dimethylsulfoxide (DMSO). However, the preferred stabilizer for the enzyme is a polyol, particularly glycerol, which is present in the range of 10% to 80% by weight based on the weight of the cell suspension.

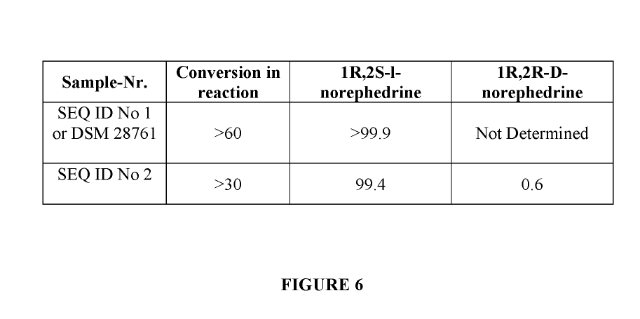

FIG. 6 illustrates the yield of the 1R, 2S-Norephedrine. After completion of the reaction, the yield of 1R, 2S-Norephedrine is determined in the presence of transaminase of SEQ ID NO: 1 or DSM 26761 in comparison with SEQ ID NO: 2. The results showed that the de % of 1R, 2S-1-norephedrine in presence of transaminase of SEQ ID NO: 1 or DSM 26761 is greater than 99.9% in comparison with the de % of 1R, 2S-1-norephedrine in presence of transaminase of SEQ ID NO: 2 which is 99.4%.

The enantioselective transamination process is cost-effective and environment-friendly in addition to providing the amine in high yield and with high enantioselectivity. The transamination using whole cells further provides the possibility of using clarified biotransformation broth containing (1R) 1-hydroxy-1phenylacetone, thereby saving in costs of isolation and making the process industrially attractive.

The present invention is further explained by the following examples:

### Example 1

### Preparation of Enzyme

Transaminase is expressed in an expression vector and is introduced in a suitable host organism, which is capable of expressing the desired gene. The preferred organism for the expression of transaminase is E. coli. E. coli is a rod shaped Gram-negative bacterium with a peritrichous flagella and is a facultative anaerobic in nature. The nucleotide sequence of the transaminase is expressed in an expression vector system pIEP/Kan/IEP AT12, which is incorporated in E. coli. The E coli colonies transformed with the expression constructs are cultivated in 200 ml of LB medium. LB medium comprises 1% tryptone, 0.5% yeast extract and 1% Sodium chloride with 50 μg/ml of kanamycin until an optical density of 0.5 measured at 550 nm is achieved. The production medium comprises of salts, glucose and ammonia as nitrogen source. The growth is at 35° C. under aerobic conditions to reach an expression of recombinant protein is induced by adding isopropylthiogalactoside (IPTG) at a concentration of 0.1 mM. After 16 hours of induction at 25° C. and 220 rpm, the cells are harvested centrifuged to 50% w/v concentration and frozen at 0-5° C.

Transaminase cell suspension is prepared by suspending 20 ml of E. coli cells in 100 ml of 100 mM potassium phosphate buffer at pH 8, prepared and stored at 05°-5° C. The cell lysates are prepared by suspending 20 ml of suspension of cells in 100 ml of 1M potassium phosphate buffer at pH 7 containing 0.1 M ethylenediaminetetraacetic acid (EDTA), phenylmethylsulfonyl fluoride (PMSF) 0.05M, dichlorodiphenyltrichloroethane (DDT) 0.5M and homogenized using a ultrasonic homogenizer. Subsequently, the enzyme solution is mixed with 200 ml of glycerol and stored at −20° C.

### Example 2

### Transamination of (1R)-1-hydroxy-1-phenylacetone

In a three-neck 500 ml round bottom flask with overhead stirring facility mounted on a water bath (25° C.-35° C.), 10 ml of 5.4 M isopropylamine is taken. To this, 40 mg of pyridoxal phosphate as a cofactor is added stirred continuously. 20 ml of 50% biomass slurry is carefully transferred and biomass is allowed to equilibrate with the cofactor and the amine donor.

In case of concentrated (1R) 1-hydroxy-1phenylacetone, the volume is made up to 100 ml with water and 0.5 gm of (1R) 1-hydroxy-1phenylacetone is added and the pH is adjusted to 8 with 3 M potassium hydroxide. This stage allows the conversion rate of greater than 99%. After 3 hours, another 0.5 gm of (1R) 1-hydroxy-1phenylacetone is added and a final 0.5 gm is added at log of 8 hours. The stirring is continued for 24 hours to allow overall conversion of greater than 90%. The de % is greater than 99%.

In case of use of biotransformation broth containing (1R) 1-hydroxy-1phenylacetone, 100 ml broth containing 9 gm/L substrate is added at log 0, pH is adjusted to 8 and after 2 hours another 80 ml of broth is added. Subsequently after another 4 hours, 60 ml of broth is added resulting in cumulative 240 ml of biotransformation broth. The stirring is continued for 24 hours to allow the overall conversion of greater than 87%. The de % is greater than 99%.

### Example 3

### Production of 1R, 2S-Norephedrine

The large scale production of an enzyme is carried out in fermenters under controlled conditions. The biomass yield obtained is about 160 gm/L wet weight. The reaction is carried as per requirement in stirred tank reactor. After reaction, the reaction mass is centrifuged to remove biomass and the clarified supernatant is taken for counter current extraction with toluene in a packed bed column. The extract is distilled in a falling film evaporator to obtain crude 1R, 2S-Norephedrine. This product is further purified by isolating as oxalate adduct by crystallization. The overall recovery is about 85% i.e. broth to oxalate. The oxalate salt is converted to hydrochloride salt or other derivatives as desired. The de % at oxalate stage is greater than 99%.

The reaction mixture is analyzed for the presence of 1R, 2S-1-Norephedrine. The de % of 1R, 2S-Norephedrine in the presence of SEQ ID NO: 1 or DSM 26761 is greater than 99.9%.

Therefore, the present invention features an enzymatic method of producing optically active chiral amines by utilizing transaminase enzyme in the presence of defined amino donor.

The enzymatic method of enantioselective amination of (1R)-1-hydroxy-1-phenylacetone to a chiral amine 1R,2S-Norephedrine in the presence of an amino donor isopropylamine catalyzed by enantioselective transaminase results in high yield of the product and is cost-effective.

We claim:

1. A method for an enzymatic synthesis of a chiral amine, the method comprising the steps of:
a. adding an amine donor to a cofactor and stirring to form a reaction mixture;
b. adding a transaminase in a completely purified form or partially purified form or plurality of cells expressing transaminase to the reaction mixture and stirring at 25° C.-35° C.;
c. adding a substrate (1R) 1-hydroxy-1-phenylacetone at a concentration of 0.5-2.5% w/v as a concentrate or as a biotransformation broth;
d. adjusting the pH to 8 with 3M potassium hydroxide; and
e. allowing the reaction for a period of 20-28 hours to result in the formation of 1R, 2S-Norephedrine and a ketone from the consumed isopropylamine.

2. The method as claimed in claim 1, wherein the amino donor is 5.4 M isopropylamine at a concentration of 10% v/v in a reaction mass.

3. The method as claimed in claim 1, wherein the co-factor is pyridoxal phosphate at a concentration of 200-400 ppm.

4. The method as claimed in claim 1, wherein transaminase is added as E. coli cells at a concentration of 10-12% w/v wet basis in the reaction mixture.

5. The method as claimed in claim 1, wherein the phosphate buffer comprises of glycerol at a concentration of 10% to 80% by weight as stabilizer.

6. The method as claimed in claim 1, wherein the temperature of the reaction mixture is maintained in the range of 20° C. to 40° C. preferably at 25° C.

7. The method as claimed in claim 1, wherein the ketone produced is acetone.

8. The method as claimed in claim 1, wherein the rate of conversion is greater than 90% and de % is greater than 99% in case of concentrated (1R) 1-hydroxy-1-phenylacetone and the rate of conversion is greater than 87% and de % greater than 99% in case of biotransformation broth.

9. The method as claimed in claim 1, wherein transaminase is a polypeptide sequence isolated from Rhodobacter sphaeroides.

10. The method as claimed in claim 1, wherein transaminase is a recombinant product expressed in E. coli through an expression vector system.

11. The method as claimed in claim 10, wherein the recombinant expression vector system is pIEP/Kan/IEP AT12.

12. The method as claimed in claim 10, wherein transaminase nucleotide sequence expressed in pIEP/Kan/IEP AT12 vector system is SEQ ID NO: 1 or DSM 26761.

13. The method as claimed in claim 1, wherein the catalysis by SEQ ID NO: 1 or DSM 26761 yields greater than 99.9% of 1R, 2S-1-Norephedrine.

14. The method as claimed in claim 1, wherein the method results in the production of stereospecific 1R, 2S-Norephedrine.
